(19) United States
(12) Patent Application Publication
(10) Pub. No.: US 2008/0220107 A1
(43) Pub. Date: Sep. 11, 2008

(54) NASAL SPRAY APPARATUS

(75) Inventor: Ake Akerman, Goteborg (SE)

Correspondence Address: O''SHEA GETZ P.C., 1500 MAIN ST. SUITE 912, SPRINGFIELD, MA, 01115, US

(73) Assignee: PHARMACURE HEALTH CARE AB, VASTRA FROLUNDA (SE)

(21) Appl. No.: 12/105,337

(22) Filed: Apr. 18, 2008

Related U.S. Application Data
(63) Continuation-in-part of application No. 10/526,455, filed on Aug. 31, 2005.

(30) Foreign Application Priority Data
Sep. 3, 2002 (EP) PCT/EP2002/009810

(51) Int. Cl. A61K 36/53; A61M 11/00

(52) U.S. Cl. 424/776; 128/200.23

(57) ABSTRACT

A nasal spray comprises a container holding a nasal preparation containing sesame oil and a spray pump dispenser mounted to close an opening of the container. A dip tube from the dispenser extends down into the nasal preparation. The pump body is provided with a mounting collar operable to create an airtight seal with the top of the container. The nasal spray further comprises an inert substance covering a surface of the nasal preparation disposed within the container that is not contiguous with a wall of the container.

### CROSS-REFERENCES TO RELATED APPLICATIONS

The present application is a continuation-in-part of U.S. patent application Ser. No. 10/526,455 filed Aug. 31, 2005.

### BACKGROUND OF THE INVENTION

1. Field of the Invention

The present invention relates to nasal sprays and apparatus for containing nasal sprays in general, and to nasal sprays of, or including sesame oil and apparatus for storing such sprays for extended periods of time in particular.

2. Background Information

It has been found that preparations including sesame oil, and in particular preparations comprising substantially pure sesame oil, can be used very successfully for the treatment of nasal discomfort, and in particular nasal dryness. Such a product is available commercially from the Applicant under the trade name Nozoil.

Nasal spray preparations have to date been administered by means of a hand operated applicator which sprays the preparation into the upper parts of the nasal cavity. The applicator includes a container for holding the preparation and a pump which is mounted by click fitted onto the top of the container containing the preparation. The prior art applicators for holding preparations containing sesame seed of which we are aware may be liquid-tight for keeping the preparation within the container, but are not air-tight; i.e., over time air can enter the container and interact with the preparation.

Some regulations limit the amount of peroxide that can be present within a pharmaceutical vegetable oil such as sesame seed oil during production of a consumer product such as a nasal preparation. Investigations to date have determined that sesame oil degrades (and creates peroxide) with exposure to air, and the degradation can be appreciable over an extended period of time. This means that the preparation stored within such a prior art applicator will likely not have an acceptable shelf life; e.g., perhaps only 6 months. It will be appreciated that the preparation may only be used occasionally, and ideally the preparation should have a shelf life of at least two years, and preferably at least three years.

A reason for the degradation in the shelf life of the nasal preparation is the manner in which the applicator is fitted onto the container holding the preparation. Another reason for the degradation in the shelf life of the product is the process by which the components of the product are assembled.

What is needed, therefore, is a nasal spray apparatus that can be used with nasal spray preparations containing sesame seed oil, which apparatus overcomes the aforesaid problems.

### BRIEF SUMMARY OF THE INVENTION

The present invention provides a nasal spray comprising a open mouthed container containing a nasal preparation containing sesame oil, and a spray pump dispenser mounted to close the open top of the container. The pump dispenser includes a dip tube sized to extend down into the preparation. The pump body dispenser includes a mounting collar which seals to the top of the container in an airtight manner; i.e., the collar seals to the top of the container sufficiently to prevent the transfer of gas between the container interior and the ambient atmosphere outside the container. The nasal spray further comprises an inert substance that covers the surface of the nasal preparation disposed within the container that is not contiguous with a wall of the container; e.g., when the nasal spray container is positioned to stand upright, the inert substance covers the upper surface of the nasal preparation disposed within the container.

In this way, the admission of air to the contents of the container is limited when the spray is not being used, and contact between any air that is admitted to the container and the preparation is limited by the presence of the inert substance that covers the preparation, leading to an improved shelf life.

In the preferred embodiment, the mounting collar is formed of a deformable material, for example aluminium which can be deformed around a flange provided around the mouth of the container to provide an airtight give an airtight closure.

Preferably a gasket is provided between the top of the container and the collar so as to ensure the seal.

Most preferably the top surface of the container around its mouth is provided with a raised annular rib which cooperates with the gasket to ensure a seal.

The body parts of the pump may be made substantially of plastics materials and the mounting collar of the pump be configured so as to hold the pump components together.

In order to reduce damage to the container contents due to light, the container is preferably made from coloured glass or plastics material.

### BRIEF DESCRIPTION OF THE DRAWINGS

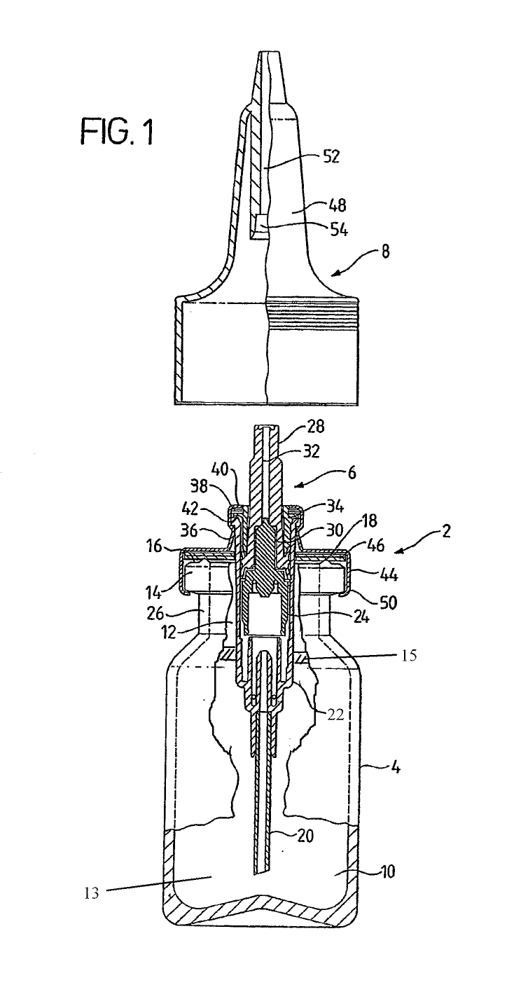

A preferred embodiment of the invention will now be described with reference to the accompanied drawings in which to FIG. 1 shows a partially exploded view of a spray according to the invention.

### DETAILED DESCRIPTION OF THE INVENTION

With reference to FIG. 1, a spray dispenser 2 comprises a container 4, a pump unit 6, a pump actuator 8 and a cap (not shown) which fits over the pump actuator 8. A nasal preparation 10 is disposed within the container 4. The preparation 10 in the preferred embodiment consists of substantially pure sesame oil. Most preferably, the sesame seed oil should be of pharmaceutical quality; i.e., refined to eliminate impurities that can start degradation as well as metals and toxic products, and to eliminate proteins that may create allergic reactions.

The container 4 is made from a colored glass so as to protect the nasal preparation 10 from light, and has a mouth 12 for receiving the pump unit 6. The mouth 12 (also referred to as the opening) of the container 4 provides access to an interior cavity 13 of the container. A mounting flange 14 for the pump unit 6 is provided around the mouth 12. The upper surface 16 of the flange is provided with a generally triangular sectioned rib 18.

An example of an acceptable pump unit 6 is of a type VP7 available commercially from Valois Division Pharmacie, which comprises a dip tube 20 that extends down into the preparation 10 within the container 4, a main body 22 of plastics such as polypropylene, a valve 24 mounted within the body 22 a spring 26 and a stem. The stem 28 has a lower bore portion 30 which receives the valve 24, and a dispensing bore 32 extending therethrough. The stem 28 and valve 24 are slidable within the body 22, and a stop member 34 limits the upward movement of the stem 28.

The body 20 and the stop 34 are held together by a deformable aluminium collar 36. The mounting collar 36 is operable to create an airtight seal between the pump unit 6 and the container 4; i.e., the collar 36 operates to seal the top of the container sufficiently to prevent the transfer of gas between the container interior 13 and the ambient atmosphere outside the container 4. A gasket 38 is provided between the upper flange 40 of the collar 36 and an opposing flange 42 of the stop member 34.

The collar 36 also mounts the pump unit 6 on the bottle 4. To this end, the collar 36 comprises a lower portion 44 which extends down and is deformed around the flange 14 of the bottle 4. A gasket 46 is positioned between the lower portion 44 of the collar 36 and the upper surface of the bottle flange 12. The annular rib 18 provided around the bottle mouth 12 engages with the gasket 46 to help ensure an airtight seal between the collar 38 and the container 4.

The actuator 8 comprises a body 48 which is received over the outer surface 50 of the pump unit 6. The body 48 is provided with an internal bore 52 having an enlarged lower end 54 which fits over and engages the upper end of the stem 30.

The cap (not shown) will have suitable means for retaining the cap on the actuator 8.

In a preferred embodiment there an inert substance 15 disposed within the container 4 to cover the surface of the nasal preparation 10 disposed within the container 4 that is not contiguous with a wall of the container 4; e.g., when the nasal spray container 4 is positioned to stand upright, the inert substance 15 covers the upper surface of the nasal preparation 10 disposed within the container 4. Preferably, the inert substance 15 comprises an inert gas such as nitrogen gas, helium gas, argon gas, or the like. The inert substance 15 may, however, comprise an inert liquid and/or may comprise both inert liquid and inert gas. The inert substance 15 disposed in the container 4 above the preparation 10 is effective to further isolate the nasal preparation 10 from the air surrounding the nasal spray apparatus 2. Furthermore, the inert substance is effective at isolating the nasal preparation 10 from the surrounding air irrespective of whether the spray apparatus 2 is upright, upside down, on its side or in any other position. Preferably, the inert substance substantially fills the entire portion of the open mouthed container 4 that is not filled with the nasal preparation. For example in a preferred embodiment, the surface of the preparation 10 is covered up to the rim of the container 4 by the inert substance.

To use the spray apparatus 2, a user inserts the actuator 8 into a nostril and presses the actuator 8 and container 4 toward each other. This actuates the pump unit 6 to dispense a predetermined dose of the preparation 10 into the user's nostril. Prior to first use of the spray 2, pressing the actuator 8 and container 4 toward each other one or more times dispenses the inert substance 15 such that the sesame oil can subsequently be dispensed into a nostril.

It will be appreciated that the collar mounting arrangement disclosed reduces the seepage of air into the container 4 when the spray apparatus 2 is not being used and presence of the inert substance 10, preferably substantially entirely filling the portion of the container 4 that does not contain the preparation 10, reduces degradation of the preparation, thereby improving the shelf life of the preparation 10.

Although this invention has been shown and described with respect to the detailed embodiments thereof, it will be understood by those skilled in the art that various changes in form and detail thereof may be made without departing from the spirit and the scope of the invention.

What is claimed is:

1. A nasal spray comprising an open mouthed container containing a preparation containing sesame oil and a spray pump dispenser mounted to close the open mouth of the container and having a dip tube extending down into the preparation, wherein the spray pump dispenser is provided with a mounting collar which seals the container in an airtight manner and the nasal spray further comprises an inert substance covering an upper surface of the preparation contained in the container.

2. The nasal spray of claim 1, wherein the preparation containing sesame oil comprises substantially pure sesame oil.

3. The nasal spray of claim 1, wherein the mounting collar is formed of a deformable material which is deformed around a flange around the mouth of the container.

4. The nasal spray of claim 3, wherein the deformable material is aluminium.

5. The nasal spray of claim 3, wherein a gasket is provided between the top of the container and the collar.

6. The nasal spray of claim 5, wherein the surface of the container around its mouth is provided with a raised annular rib which cooperates with the gasket.

7. The nasal spray of claim 1, wherein the body parts of the spray pump dispenser are held together by the mounting collar.

8. The nasal spray of claim 1, wherein the container is made from a coloured glass or plastics material.

9. The nasal spray of claim 1, wherein the inert substance comprises an inert gas.

10. The nasal spray of claim 9, wherein the inert gas is nitrogen.

11. The nasal spray of claim 1, wherein the inert substance substantially fills the entire portion of the open mouthed container that is not filled with the preparation.

12. A nasal spray apparatus, comprising:
a nasal preparation containing sesame seed oil;
a container having an opening that provides access to an interior cavity, which container is operable to hold the nasal preparation within the interior cavity;
a spray pump dispenser having a dip tube and a mounting collar, which spray pump dispenser is mounted on the container in a manner that closes the opening;
wherein the mounting collar is operable to seal the interior cavity of the container sufficiently to prevent a transfer of gas between the interior cavity and an ambient atmosphere outside the container; and
an inert substance covering a surface of the nasal preparation disposed within the container that is not contiguous with a wall of the container.

13. The nasal spray apparatus of claim 12, wherein the mounting collar is formed of a deformable material which is deformed around a flange around the opening of the container.

14. The nasal spray apparatus of claim 13, wherein a gasket is provided between the container and the mounting collar.

15. The nasal spray apparatus of claim 14, wherein a surface of the container around the opening mouth includes a raised annular rib that engages the gasket.

16. The nasal spray apparatus of claim 12, wherein the inert substance comprises an inert gas.

17. A method for storing a nasal spray preparation, comprising the steps of:
providing a nasal preparation containing sesame seed oil;
disposing the nasal preparation within an interior cavity of a container, which container has an opening that provides access to the interior cavity;
mounting a spray pump dispenser having a dip tube and a mounting collar on the container in a manner that closes the opening;
covering a surface of the nasal preparation disposed within the container that is not contiguous with a wall of the container with an inert substance; and
sealing the interior cavity of the container sufficiently to prevent a transfer of gas between the interior cavity and an ambient atmosphere outside the container.

18. The method of claim 17, wherein the inert substance is an inert gas.

19. The method of claim 18, wherein the covering step includes substantially filling a remainder of the open mouthed container that is not filled with the preparation, with the inert gas.
